(19) United States
(12) Patent Application Publication
(10) Pub. No.: US 2022/0023140 A1
(43) Pub. Date: Jan. 27, 2022

(54) DEVICE FOR TREATMENT ERECTILE DYSFUNCTION

(71) Applicant: Inmode Ltd., Yokneam (IL)

(72) Inventor: Michael Kreindel, Richmond Hill (CA)

(73) Assignee: Inmode Ltd., Yokneam (IL)

(21) Appl. No.: 16/938,992

(22) Filed: Jul. 26, 2020

(51) Int. Cl. A61H 19/00; A61H 9/00; A61N 5/06

(52) U.S. Cl. CPC A61H 19/32; A61H 9/0057; A61N 2005/067; A61H 9/0007; A61H 2201/0207; A61N 5/0625

(57) ABSTRACT

The invention relates to a method for treatment of erectile dysfunction using a combination of heat and negative pressure.

### FIELD OF THE INVENTION

The invention relates to a device for treatment erectile dysfunction creating revascularization in penis using negative pressure and deep heating.

### BACKGROUND OF THE INVENTION

Erectile dysfunction is a common problem for aged male individuals. The most popular solution for these patients is PDE5 inhibitors. However, not everyone responds to the medication and not everyone is satisfied with the solution because of lack of spontaneity.

An alternative solution approved by the FDA and recommended by leading urological centers is a vacuum pump with constricting ring. The device is described in U.S. Pat. No. 4,378,008. The device includes an elongated vacuum cylinder which receives the male organ and which is connected by means of a flexible conduit to a vacuum source by which the interior of the cylinder may be evacuated to cause the organ to become erect and distended A manually operated valve is connected in the flexible conduit which may be easily closed to maintain the partial vacuum and organ erect in the cylinder while an elastic band carried adjacent the open end of the cylinder is fitted over the base of the organ. The valve may then be opened to relieve the vacuum and remove the vacuum cylinder with the erection being captured. Usually, constriction is applied for no longer than 30 minutes because of safety reasons related to terminating of blood circulation in the organ.

Other patents that describe similar methods include U.S. Pat. Nos. 6,458,073, 5,344,389 and 5,344,389.

U.S. Pat. No. 7,083,570 describes a specific vacuum device for use in the treatment of erectile dysfunction or for penile exercise.

U.S. Pat. No. 7,037,257 describes a method for treatment of erectile dysfunction in which a vacuum device is combined with PDE5 inhibitor treatment.

Another method of treatment is based on shock wave pulses to the penis to stimulate tissue re-vascularization, such as U.S. Pat. Nos. 10,441,498 and 9,913,748.

### SUMMARY

The present invention describes a method for treating erectile dysfunction with a combination of vacuum (negative pressure) and thermal therapy.

In some embodiments the negative pressure is created in a chamber to stretch the tissue and open blood vessels. The negative pressure can be created by a vacuum pump connected to the chamber, by increasing chamber volume or by connecting the treatment chamber to another volume having lower pressure.

Prolonged application will pump blood into the penile tissue and open blood vessels. To get longer lasting effect, the application of negative pressure may be applied for a longer time during a multiple treatment. Application of too strong negative pressure for a long time may create excessive expansion of blood vessels resulting in tissue bruising. To avoid this effect, pressure and application time may be controlled. A vacuum sensor may be connected to the treatment chamber to monitor the vacuum level.

Vacuum can be applied in a continuous wave (CW) or a pulsed manner Multiple regimes (combinations of CW and pulses) can be programmed by the user or preset by the manufacturer of the device. Long application of vacuum allows deeper penetration of the negative pressure into the tissue. The pulsed application of negative pressure may safely achieve higher vacuum amplitudes.

The treatment chamber has a cylindrical shape and is large enough to accept the erected penis. The chamber may have different shapes that enable sealing against the penile tissue and permit the use of different connected accessories. The typical chamber size, without limitation, may have a length of 20 cm and a diameter of 6 cm, which provides a chamber volume of 565 cc.

To be able to work in pulse mode with a pulse width up to 10 sec, the vacuum pump may provide an air flow of about 20-100 L/min High flow of the pump will allow better sealing of the chamber to the tissue. For CW operation 3-10 L/min is enough to maintain the required vacuum. Vacuum level in CW operation may be in the range −100 mbar to −300 mbar to avoid bruising. The invention is not limited to these values or any of the values described in the description.

For a pulsed device, the vacuum level can be up to −600 mBar. The shorter vacuum pulse permits applying a higher vacuum level.

The vacuum pump can be located in an applicator or in the console and connected to the chamber. The pump can be connected with the treatment chamber through the vacuum valve for fast termination of vacuum suction.

Another valve can be used to restore atmospheric pressure. Alternatively, there may be a constant small leakage of air to the treatment chamber. Leakage of air should be significantly lower than air flow created by vacuum pump.

In other embodiment, a pressure pump can be used to create high pressure in the treatment chamber and alternate negative and positive pressure pulses.

The invention uniquely combines the vacuum with thermal energy to synergistically increase blood circulation. The heating makes tissue softer that decrease blood vessel compression. The other effect is a physiological reaction arising from a deviation from normal temperature. To maximize the effect, the heat may penetrate deep in to the tissue. RF energy has unlimited penetration depth but attaching it firmly to the penile tissue, which changes shape and volume during the treatment, is quite a challenge. Additionally, in order to avoid cross-contamination between patients the parts contacting the tissue should be disposable, which makes electrode contacting system costly and complex.

An alternative method is to use optical energy which can be delivered without contacting the tissue. The optical energy can be generated using a laser, LED, filament lamp or gas-filled lamp.

Optical energy should ideally penetrate to a depth of 1 cm or more, but in the real world the best penetration depth for optical energy in the red and near infrared range is from 600 nm to 1200 nm. This range of light is less sensitive to skin pigmentation.

Lamps having broad light spectrum may be filtered to optimize safety and penetration depth.

The light energy can be delivered in CW, quasi-CW or in a pulsed mode. CW energy allows uniform heating of tissue; pulses allow heating blood vessels selectively. The light spectrum from 600 nm to 1200 nm is absorbed selectively by hemoglobin; if the pulse width is shorter than the typical thermal relaxation time of the blood vessels, then the vessels can have a higher temperature than the surrounding tissue.

The diode laser and LED are the preferred solution because of following reasons: a) high efficiency (diode laser has efficiency about 50% and LED about 30%), b) small size of LED and diode lasers allows compact design of applicator, and c) high availability and efficiency in the required spectral range. An advantage of lamps is the low cost of the light source.

The average power density of optical energy may be in the range of 0.1-2 W/cm².

The light source may be cooled by water or forced air to dissipate heat from the applicator. The light source may provide uniform heating of the entire area of the treated tissue.

The multiple heating sources may be arranged radially around the penis. The vacuum chamber may be made from a material transparent for the optical energy. In this case, heating elements should not be in contact with treated tissue.

Alternatively, other heating sources including ultrasound, RF, microwave can be used.

In order to avoid tissue burn and provide effective thermal treatment, a temperature sensor can be used in a feedback control loop with the controller. The sensor can be, without limitation, a thermistor or thermocouple. Alternatively, an infrared thermal sensor can be used to monitor tissue temperature. The preferred tissue heating is in the range of 38-45° C. The heating should be strong enough to create tissue erythema but not cause thermal necrosis of the tissue.

There is provided in accordance with a non-limiting embodiment of the invention a method for treating erectile dysfunction including applying negative pressure to penile tissue while heating the penile tissue with thermal energy, wherein a combination of the negative pressure and the thermal energy improves blood flow in the penile tissue to treat erectile dysfunction of the penile tissue, wherein the negative pressure is below ambient pressure and the penile tissue is heated to a heated temperature above body temperature.

The thermal energy may be applied from a source of optical energy, which may be a laser, a light emitting diode, a filament lamp, or a gas-filled lamp, for example.

The negative pressure may be pulses of negative pressure or continuous application of negative pressure. The heated temperature may not exceed a coagulation temperature of the penile tissue, such as not exceeding 45° C.

There is provided in accordance with a non-limiting embodiment of the invention a method for treating erectile dysfunction including applying alternating negative and positive pressure to a penile tissue while heating the penile tissue with thermal energy, wherein a combination of the alternating negative and positive pressure and the thermal energy improves blood flow in the penile tissue to treat erectile dysfunction of the penile tissue, wherein the negative pressure is below ambient pressure and the positive pressure is above ambient pressure and the penile tissue is heated to a temperature above normal body temperature.

There is provided in accordance with a non-limiting embodiment of the invention a method for treating erectile dysfunction including stretching penile tissue while heating the penile tissue with thermal energy, wherein a combination of the stretching and the thermal energy improves blood flow in the penile tissue to treat erectile dysfunction of the penile tissue, wherein the stretching opens compressed blood vessels and the penile tissue is heated to a heated temperature above normal body temperature. The stretching may be created by applying negative pressure to the penile tissue.

### BRIEF DESCRIPTION OF THE DRAWINGS

In the accompanying drawings:

FIG. 1 is a schematic illustration of a device, in accordance with a non-limiting embodiment of the invention, attached to the patient.

FIG. 2 is a schematic illustration of an applicator in accordance with a non-limiting embodiment of the invention.

FIG. 3 is a schematic illustration of a vacuum system in accordance with a non-limiting embodiment of the invention.

FIG. 4 is a schematic illustration of a disposable vacuum chamber in accordance with a non-limiting embodiment of the invention.

FIG. 5 is an illustration of light penetration depth in a preferred (but non-limiting) spectrum.

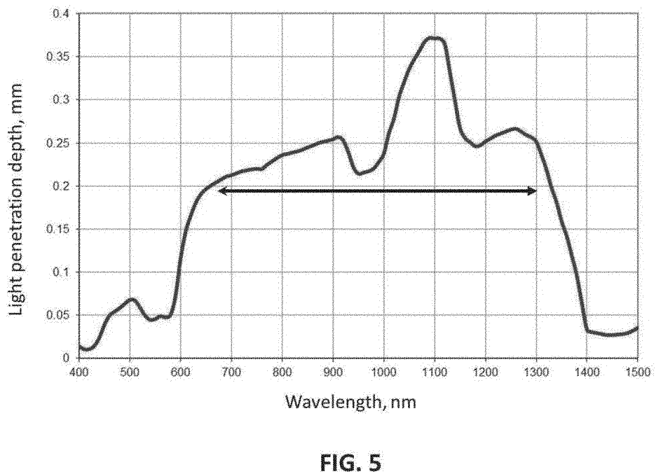

### DETAILED DESCRIPTION

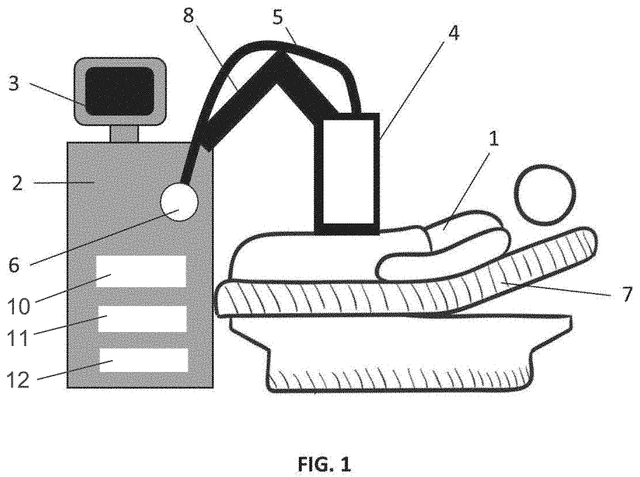

Referring to FIG. 1, a device 2 includes an applicator 4, which is applied to a penis of a patient 1. Device 2 includes a controller 10, one or more power supplies 11, a vacuum pump 12, and a display 3 (with a user interface, such as a touch panel or keyboard and the like). The applicator 4 is coupled to device 2 with an articulated arm 8, which is used to position applicator 4 on patient 1. Applicator 4 may be connected to the controller 10, power supply 11, and vacuum pump 12 of device 2 via a harness 5 and a connector 6.

The patient 1 lies or sits on a bed 7 during the treatment. The treatment attendant can apply applicator 4 to the penis, set treatment parameters and activate the treatment. The articulated arm 8 maintains the position of applicator 4 during the treatment, which can last from 15-90 minutes.

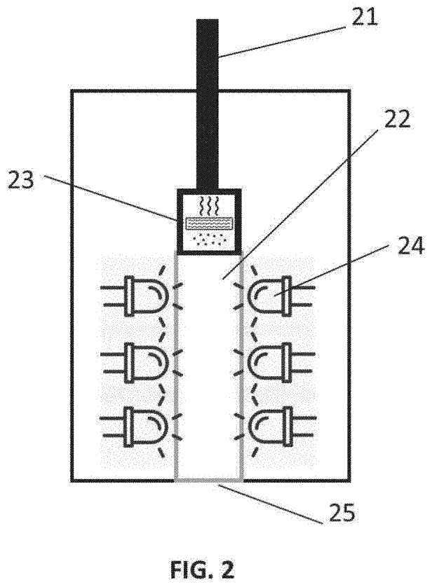

FIG. 2 illustrates structure of applicator 4, which includes a vacuum chamber 22 with an opening 25. The penis is inserted into chamber 22 through opening 25 and is attached to the patient with a firm pressure to seal vacuum chamber 22. Vacuum chamber 22 may be made from plastic transparent for visible and near infrared light. One or more light sources 24 irradiate the penile tissue radially to heat the penile tissue. Vacuum suction, i.e., the negative pressure created by the vacuum pump 12 (FIG. 1), may be communicated to chamber 22 through a tube 21 and air filter 23.

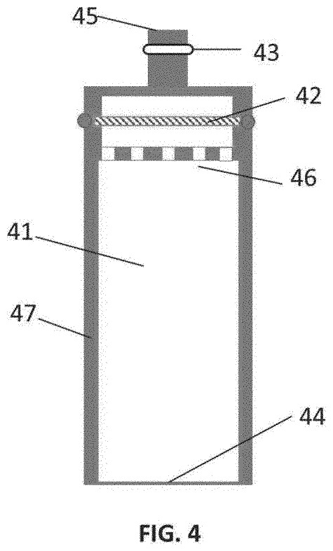

Another version of a disposable vacuum applicator is shown in FIG. 4. This vacuum applicator includes a cylinder 47 made from transparent plastic with an opening 44 for inserting the penis into vacuum chamber 41. Vacuum suction is performed through the inlet 45 connected to the applicator and sealed with a silicone O-ring 43. The air passes through a filter 42 separated from the vacuum chamber 41 with a grid 46. A rubber ring may be applied at opening 44 of the vacuum chamber 41 for better sealing between vacuum chamber 41 and attached tissue.

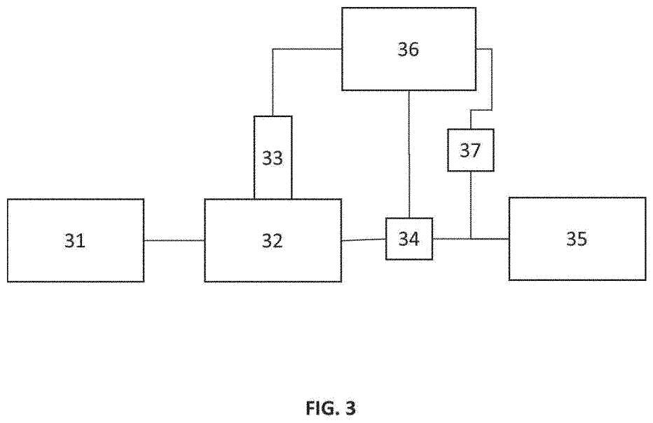

FIG. 3 is a block diagram of one version of the vacuum pump for use in the device. The vacuum pump 31 is connected to a reservoir 32 having a much larger volume than a vacuum chamber 35 in the applicator. Vacuum pump 31 is used to create the required vacuum in reservoir 32. A vacuum sensor 33 may be used to monitor the vacuum level in the reservoir 32, and may be used to maintain the vacuum at a predetermined level. Controller 36 monitors input from vacuum sensor 33 and activates vacuum pump 31 accordingly to maintain the vacuum level. In order to apply vacuum pulses to the tissue, controller 36 may open valve 34 that connects reservoir 32 and vacuum chamber 35. To release vacuum in the treatment chamber 35, the vacuum valve 34 is closed and pressure valve 37 is opened allowing air with atmospheric pressure to fill the treatment chamber 35.

Non-limiting parameters for the device of the invention are as follows:

1. Vacuum level applied to the tissue is in the range of −100 to −400 mbar.

2. Vacuum may be applied with pulses having duration from 1 to 30 sec.

3. The optical energy for heating the tissue may be in the wavelength range of 600 to 1300 nm.

4. The optical energy source may be an LED or diode laser.

5. The power density of optical energy on the tissue surface may be 0.1 to 5 W

6. Tissue heating 38-45° C. The heated temperature does not exceed a coagulation temperature of the penile tissue.

In contrast to the above described invention, different combinations of thermal and mechanical energies may be used, for example, combinations of heat and mechanical forces generated by shock wave energy, ultrasonic energy and/or vibration energy may be used.

1. A method for treating erectile dysfunction comprising:
applying negative pressure to penile tissue while heating said penile tissue with thermal energy, wherein a combination of said negative pressure and said thermal energy improves blood flow in said penile tissue to treat erectile dysfunction of said penile tissue, wherein said negative pressure is below ambient pressure and said penile tissue is heated to a heated temperature above body temperature.

2. The method according to claim 1, wherein said thermal energy is applied from a source of optical energy.

3. The method according to claim 2, wherein said source of optical energy is a laser.

4. The method according to claim 2, wherein said source of optical energy is a light emitting diode.

5. The method according to claim 2, wherein said source of optical energy is a filament lamp.

6. The method according to claim 2, wherein said source of optical energy is a gas-filled lamp.

7. The method according to claim 1, wherein the negative pressure comprises pulses of negative pressure.

8. The method according to claim 1, wherein the negative pressure comprises continuous application of negative pressure.

9. The method according to claim 1, wherein said heated temperature does not exceed a coagulation temperature of said penile tissue.

10. The method according to claim 1, wherein said heated temperature does not exceed 45° C.

11. A method for treating erectile dysfunction comprising:
applying alternating negative and positive pressure to a penile tissue while heating said penile tissue with thermal energy, wherein a combination of said alternating negative and positive pressure and said thermal energy improves blood flow in said penile tissue to treat erectile dysfunction of said penile tissue, wherein said negative pressure is below ambient pressure and said positive pressure is above ambient pressure and said penile tissue is heated to a temperature above normal body temperature.

12. A method for treating erectile dysfunction comprising:
stretching penile tissue while heating said penile tissue with thermal energy, wherein a combination of said stretching and said thermal energy improves blood flow in said penile tissue to treat erectile dysfunction of said penile tissue, wherein said stretching opens compressed blood vessels and said penile tissue is heated to a heated temperature above normal body temperature.

13. The method according to claim 12, wherein said stretching is created by applying negative pressure to said penile tissue.
